(19) United States
(12) Patent Application Publication
(10) Pub. No.: US 2015/0240240 A1
(43) Pub. Date: Aug. 27, 2015

(54) MSF REPROGRAMS MYOFIBROBLASTS TOWARD LACTATE PRODUCTION AND FUEL ANAERBOIC TUMOR GROWTH

(71) Applicants: Michael P. LISANTI, (US); THOMAS JEFFERSON UNIVERSITY, Philadelphia, PA (US)

(72) Inventor: Michael P. Lisanti, Manchester (GB)

(73) Assignee: Thomas Jefferson University, Philadelphia, PA (US)

(21) Appl. No.: 14/427,549

(22) PCT Filed: Sep. 13, 2013
(86) PCT No.: PCT/US13/59679

§ 371 Date: Mar. 11, 2015

Related U.S. Application Data
(60) Provisional application No. 61/700,777, filed on Sep. 13, 2012.

(51) Int. Cl. C12N 15/113

(52) U.S. Cl. CPC C12N 15/1137; C12N 15/113; C12N 2310/11

(57) ABSTRACT

Presented herein is a method for treating a subject afflicted with a tumor comprising administering to the subject a therapeutically effective amount of an agent that inhibits the MSF/Cdc42/NFkB cascade in the subject's tumor-associated fibroblasts.

An important relationship exists between tumor cells and their local extrα-cellular microenvironment ¹⁻⁴. Indeed, tumor-associated stromal cells critically influence cancer progression and metastasis ¹⁻⁴. Thus, tumor progression is the product of interactions between cancer cells and adjacent stromal cells, such as immune cells, endothelia and fibroblasts, although the exact mechanism(s) still remain poorly understood ⁵⁻⁷.

More specifically, stromal myo-fibroblasts are now considered active metabolic drivers of tumor growth ⁸. In our recent studies, we proposed that stromal fibroblasts fuel epithelial tumor cells via a unilateral transfer of energy-rich nutrients, from the tumor stroma to cancer cells ⁹ . In accordance with this assertion, the recycled nutrients produced by stromal fibroblasts, via autophagy/mitophagy, provide a steady-stream of energy-rich metabolites to cancer cells, inducing mitochondrial biogenesis ¹⁰⁻¹⁵.

Normal stromal fibroblasts are converted into carcinoma-associated fibroblasts (CAFs) by complex interactions with adjacent cancer cells ^{5, 16-19}. These CAFs show a fetal-like phenotype, characterized by the expression of molecules typically expressed during embryonic development. In addition, CAFs develop a myofibroblast-phenotype, with the expression of smooth muscle cell markers and the local production of transforming growth factor β (TGF-β), which can actively spread the CAF phenotype²⁰⁻²⁶.

Fetal-like fibroblasts and myo-fibroblasts, are also both viewed as “activated fibroblasts”, due to their increased expression of both ECM components and inflammatory cytokines²⁷⁻³⁴. Fetal-like fibroblasts also secrete a soluble, genetically-truncated, form of fibronectin, termed Migration Stimulating Factor (MSF)²⁷⁻³⁴. Interestingly, MSF is highly expressed in both fetal epithelial and stromal cells and in cancer patients, but its expression is somehow suppressed in normal adults²⁷⁻³⁴.

Detailed molecular characterization of MSF indicates that it is a 70-kDa protein, which is essentially identical to the N-terminal domain of full-length fibronectin, with the addition of an MSF-specific 10 amino-acid C-terminal sequence³⁵⁻³⁶. MSF changes the behavior of many target cell populations (fibroblasts, vascular and epithelial cells), by stimulating migration/invasion, matrix remodelling and neo-angiogenesis³⁷⁻⁴⁶.

Here, we generated a new hTERT-immortalized fibroblast cell line over-expressing MSF, in order to clarify the functional role of MSF in driving the cancer-associated fibroblast phenotype. Now, we demonstrate that MSF-expressing fibroblasts create an autophagic/catabolic tumor stroma, which then provides high-energy nutrients to epithelial cancer cells, via a paracrine mechanism.

### Results

To directly assess the role of MSF in tumor growth, we stably over-expressed MSF in an immortalized human fibroblast cell line (hTERT-BJ1 cells) (FIG. 1A). Empty vector (Lv-105) control fibroblasts were produced in parallel. FIG. 1A shows that transduction with MSF lentiviral particles successfully increased the stable expression of the MSF protein.

Fibroblasts Over-Expressing MSF Develop a Cancer-Associated Fibroblast Phenotype, Characterized by the Expression of Myofibroblast Marker Proteins and Activated TGF-β Signaling.

Cancer-associated fibroblasts exhibit a myo-fibroblastic phenotype, characterized by the synthesis of intracellular smooth muscle markers, in particular α-smooth muscle actin (α-SMA). To evaluate if MSF expression promotes myo-fibroblastic differentiation, MSF-expressing fibroblasts were subjected to immuno-blot analysis, using a panel of myo-fibroblastic markers (FIG. 1A). The results show that MSF is indeed sufficient to induce the increased protein expression of SMA, Calponin (particularly, isoforms 1 and 3) and Fibronectin (full length).

Several lines of evidence indicate that activated-fibroblasts increase their expression and secretion of TGF-β, thereby promoting tumor growth. Thus, we next tested if MSF over-expression upregulates the expression of TGF-β. Consistent with this hypothesis, FIG. 1B shows that MSF-overexpressing fibroblasts are characterized by an increase in TGF-β expression and a down-regulation of its receptor, TGFβ-RI, both indicative of activated TGF-β signaling.

Fibroblasts Over-Expressing MSF Migrate to a Significantly Greater Extent than do Control Cells and they Also Function as Chemo-Attractants, Stimulating Cancer Cell Migration.

MSF is a potent motogenic factor, which is able to stimulate the migration of fibroblasts, epithelial, as well as endothelial cells ³⁵⁻⁴⁶. Here, we demonstrate that MSF over-expression stimulates the migration of fibroblasts, validating the motogenic activity of the MSF protein (FIG. 2A). MSF could also influence the migration of cancer cells, by acting on these cells as chemo-attractant. In support of this notion, FIG. 2B shows that cancer cells, in presence of MSF over-expressing fibroblasts, migrate to a greater extent (1.4 fold) than do cancer cells in presence of normal control fibroblasts.

This migratory activity could be induced by the increased expression and/or activation of proteins that play a fundamental role in cytoskeletal organization. Small GTPases, such as Rac1 and Cdc42, play a central role in regulating cell movement and migration, by interacting with other proteins that more directly confer cytoskeletal rearrangements. As predicted, FIG. 2C shows that MSF over-expression up-regulates the expression levels of both these small-GTPases, which are associated with remodeling the actin cytoskeleton.

Fibroblasts Over-Expressing MSF Activate Nfkb, Exhibit the Induction of Autophagy and Cell Cycle Arrest.

GTPases are strong activators of the transcription factor NFkB, so we next validated that MSF is able to induce not only the upregulation of Cdc42 and Rac1, but also the activation of NFkB. As shown in FIG. 3A, MSF over-expression resulted in increased levels of p-NFkB, suggesting that MSF could influence the stromal fibroblasts through the activation of a number of different signaling pathways, including the NFkB signaling pathway.

NFkB plays a pivotal role as a signal integrator, which controls the autophagic process. For this purpose, we evaluated if the activation of NFkB in stromal MSF fibroblasts is sufficient to promote the autophagic process. Therefore, fibroblasts over-expressing MSF were analyzed by immuno-blot analysis, using a panel of autophagy markers. FIG. 3B shows that MSF increases the expression of several classical autophagy markers, such as Beclin1, BNIP3 and LC3-I. These results suggest that MSF augments or activates the autophagic process in stromal fibroblasts, probably via increased activation of the NFkB pathway. This pro-autophagic phenotype is associated with cell cycle arrest, as evidenced by the up-regulation of CDK inhibitors, such as p21(CIP1/WAF1), p19(ARF) and p16(INK4A) (FIG. 3C).

Under Hypoxic Conditions, MSF-Fibroblasts Generate Elevated Levels of L-Lactate and Show Decreased Mitochondrial Activity, Consistent with a Shift Towards Glycolytic Metabolism.

We have previously shown that stromal fibroblasts promote and fuel tumor growth, via activation of an autophagic program in the tumor stroma ^{8,9, 12-15}. Autophagy leads to the generation of recycled catabolic nutrients that can be used to power the anabolic growth of cancer cells. Because L-lactate is a critical fuel which provides continued energetic support for cancer cells, we next examined if MSF-fibroblasts are able to induce L-lactate accumulation. As shown in FIG. 4A, fibroblasts over-expressing MSF display increased L-lactate production (˜2-fold; p=0.004; 1.5-fold p=0.03, values expressed as nmoles/μg or pmoles/cells, respectively). However, the ability of MSF-fibroblasts to secrete L-lactate was observed only under hypoxic conditions.

That L-lactate accumulation is indicative of a shift towards predominantly glycolytic metabolism. This observation was validated by assessing the status of mitochondrial activity in MSF-fibroblasts. FIG. 4B shows decreased mitochondrial activity, as predicted, as visualized using MitoTracker staining. Note that MSF induces a dramatic reduction in MitoTracker staining, indicative of a loss of healthy functional mitochondria, both under normoxic, as well as hypoxic conditions.

As shown in FIG. 4C, MSF over-expression leads to Akt-activation, that likely protects these cells against apoptosis. MSF-fibroblasts were subjected to immuno-blot analysis, using phospho-specific antibodies directed against different protein components of the Akt pathway.

Note that MSF induces the activation of Akt-downstream effectors, such as phospho-mTOR and phospho-p70S6 kinase, both involved in protein biosynthesis. Akt normally activates mTOR, leading to p70S6K activation. Activation of Akt-pathway by MSF in stromal fibroblasts may lead to activation of protein synthesis, as a compensatory mechanism to prevent apoptotic cell death in cells undergoing constitutive autophagy/mitophagy.

Fibroblast Over-Expressing MSF Promote Tumor Growth, without any Increases in Tumor Angiogenesis.

Because MSF fibroblasts are able to increase L-lactate production and have an strong autophagic phenotype, we evaluated whether MSF is able to promote tumor growth. For this purpose, we developed a human tumor xenograft model. MSF over-expressing fibroblasts were co-injected with MDA-MB-231 breast cancer cells into the flanks of immuno-deficient nude mice. FIG. 5A demonstrates that MSF over-expression in stromal fibroblasts is sufficient to promote tumor growth, as evidenced by significant increases in both tumor weight and volume.

Stromal expression of MSF may contribute to tumor pathogenesis by a number of mechanism(s), including the stimulation of angiogenesis. To address this issue, frozen tissue sections derived from tumor xenografts were subjected to immuno-staining with a well-established vascular marker, namely CD31. As shown in FIG. 5B, MSF over-expression in stromal fibroblasts does not have a significant effect on tumor neo-vascularization, indicating that the tumor-promoting effects of MSF in cancer-associated fibroblasts are independent of tumor angiogenesis.

SMA, Rac1 and Cdc42 Over-Expression in Fibroblasts Induces Myo-Fibroblast Differentiation.

We demonstrated above that MSF-fibroblasts show increased expression of SMA and two small GTPase proteins, namely Rac1 and Cdc42. To determine if there is a cause-effect relationship here, we employed a genetic approach by over-expressing SMA, Rac1, and Cdc42 in an immortalized human fibroblast cell line (FIG. 6A, B, C) Then, these fibroblast cell lines were subjected to immuno-blot analysis, employing a panel of myo-fibroblast markers, in order to characterize their phenotype.

Note that Rac1- and Cdc42-overexpressing fibroblasts display the up-regulation in SMA protein expression (FIG. 7A) and all three over-expressing cell lines show increases in the calponin and vimentin (FIG. 7B), consistent with a myo-fibroblast phenotype. Similarly, both GTPases, Rac1 and Cdc42, were able to induce the reorganization of the F-actin cytoskeleton, as evidenced by an increase in the density of actin stress fibers, as visualized by Phalloidin-staining (FIG. 7C); note the bundles of parallel fibers aligned along the cell axis.

Cdc42-Over-Expression Induces NFkB-Activation, with Increased Autophagy and a Shift Toward Glycolytic Metabolism.

GTPases are strong activators of the transcription factor NFkB. Thus, we evaluated the effects of expressing SMA, Rac1 and cdc42 in fibroblasts, on the status of NFkB and p-NFkB. Our results demonstrate that the p-NFkB protein levels are significantly increased only in Cdc42 over-expressing fibroblasts (FIG. 8A).

For this and all subsequent experiments, we chose to examine only the fibroblasts over-expressing SMA and Cdc42; SMA was used as a negative control and Cdc42 was used as it is the GTPase that activates NFkB. To evaluate if this Cdc42-driven NFkB-activation promotes autophagy, fibroblasts over-expressing SMA and Cdc42 were subjected to immuno-blot analysis, using a panel of autophagy markers.

FIG. 8B demonstrates that Cdc42 over-expression in fibroblasts drives the increased expression of mitophagy (BNIP3) and autophagy markers (Beclin-1, LAMP1 and Cathepsin B).

Also, we evaluated if Cdc42 over-expressing fibroblasts are able to induce L-lactate accumulation and a shift towards glycolytic metabolism. FIG. 8C demonstrates that Cdc42 expression is sufficient to induce an ˜80% increase in L-lactate production, under hypoxic condition and after treatment with Metformin, a specific inhibitor of mitochondrial complex I. This shift towards glycolytic metabolism was further validated by MitoTracker staining, showing that Cdc42 expression strongly decreases mitochondrial activity, under hypoxic conditions (FIG. 8D).

Stromal Expression of Cdc42 Promotes Increased Tumor Growth in Vivo.

To evaluate if Cdc42 expression in stromal cells is able to promote tumor growth in vivo, we used a human tumor xenograft model. Control, SMA-. or Cdc42-fibroblasts were co-injected with MDA-MB-231 breast cancer cells in the flanks of immuno-deficient nude mice. FIG. 9A shows that over-expression of Cdc42 in stromal fibroblasts consistently promotes tumor growth, over a 25 day time course. FIG. 9B shows that, at 4 weeks post-injection, Cdc42-fibroblasts increased tumor volume by ˜1.75-fold, as compared with vector-alone control fibroblasts cells, directly demonstrating that stromal Cdc42 is able to support tumor growth in vivo.

Finally, to determine the role of neo-vascularization in Cdc42-mediated tumor growth, we quantified neo-vascularization via immuno-staining with CD31 (FIG. 9C). However, a 25% increase of tumor angiogenesis in Cdc42 tumors is not sufficient to account for a near two-fold increase in tumor growth. Instead, metabolic reprogramming of the tumor microenvironment, towards L-lactate production, is a more likely mechanism.

### Discussion

The role of the host stromal microenvironment in promoting tumor initiation and progression is now well-established ¹⁻⁴. However, the exact molecular mechanism(s) of how cancer-associated fibroblasts promote tumor growth remain unknown. Here, we highlight that MSF (migration stimulating factor) functions to metabolically reprogram stromal fibroblasts towards glycolytic metabolism, resulting in the generation of a catabolic tumor micro-environment that actively “fuels” anabolic tumor growth.

More specifically, MSF over-expressing fibroblasts were used to mimic the “activated microenvironment” that is now widely known to support tumor growth. We demonstrated that MSF-fibroblasts show many characteristics of differentiated myofibroblasts, including the expression of smooth muscle-specific proteins.

Transforming growth factor-β (TGF-β) is a potent inducer of myo-fibroblast differentiation that has been implicated in conferring the tumor-associated fibroblast phenotype ^{3, 18, 19, 47-50}. Here, we have demonstrated that MSF over-expression in stromal fibroblasts leads to the increased production of TGF-β and is associated with a reduction in the expression of its receptor, TGF β-RI. The expression of TGF-β, specifically TGF-β1, is up-regulated in most tumors and seems to play a key role in cancer progression ^{3, 18, 19,47-50}.

Increased TGF-β expression in fibroblasts benefits cancer progression, likely via paracrine effects on tumor cells ^{18, 19, 47-50}. In particular, the release of TGF-β in the vicinity of cancer cells may result in a more hospitable microenvironment, facilitating tumor growth. Several authors have shown that TGF-β over-expression leads to an increased metabolic rate, due to enhanced glycolysis ^{51, 52}. MSF may induce glycolysis in stromal fibroblasts via increased endogenous production of TGF-β. The observed increase in glycolytic metabolism may be due to the autophagic destruction of mitochondria in MSF over-expressing fibroblasts. This assertion is consistent with our previous observations that autophagy in cancer-associated fibroblasts is able to generate a catabolic tumor stroma that drives the anabolic growth of cancer cells ^{8, 9}. Regardless of the exact mechanism activating glycolysis, MSF is able to produce a catabolic energy-rich microenvironment, favoring to tumor growth.

Small GTPases, such as Rac1 and Cdc42, are known to play a critical role in cell migration and invasion ^{53, 54}. However, their potential roles in myo-fibroblast differentiation, autophagy, and cellular metabolism are under appreciated, In the current study, we demonstrated that MSF over-expressing fibroblasts have increased expression both Rac1 and Cdc42. To determine whether increased Rac1 and/or Cdc42 expression influences the activation of tumor microenvironment, we generated Rac1 and Cdc42 over-expressing fibroblasts.

Our results demonstrate that both Rac1 and Cdc42 fibroblasts undergo myo-fibroblast differentiation, with characteristic re-organization of the actin cytoskeleton. However, only Cdc42-fibroblasts show activation of NFkB, with the onset of autophagy and a shift towards predominantly glycolytic metabolism in the tumor stroma, resulting in the promotion of tumor growth. Therefore, over-expression and/or activation of Cdc42 is a likely mechanism by which MSF induces NFkB-activation, leading to increased autophagy and glycolysis, due to reduced mitochondrial function. As such, glycolytic/catabolic MSF-fibroblasts create a favorable metabolic microenvironment to support tumor growth.

In conclusion, our results highlight the critical functional role of MSF as a driver of cancer progression. This is consistent with its ability to stimulate the migration/invasion in both stromal and tumor cells, and its effects on the metabolic remodeling of the tumor microenvironment.

### Materials and Methods

Materials

Reagents were purchased as follows: the specific and cell-permeable proteasome inhibitor (MG132) was from Calbiochem (San Diego, Calif.; used at a final concentration of 10 μM for 16 h); Metformin (1,1-dimethylbiguanide hydrochloride) was from Sigma (D150959); Alexa Fluor 633 Phalloidin (A222284) was from Invitrogen. Antibodies to the following target proteins were also used: Fibronectin N-terminal (Chemicon International, MAB1936; to recognize MSF); Fibronectin (Abeam, ab23750); Vimentin (BD Pharmingen, 550513); Calponin 1/2/3 (Santa Cruz Biotech, sc-28545); Smooth Muscle Actin (Dako, M0851); Beelin (Novus Biologicals, NBP1-00085); BNIP-3 (Abeam, ab10433); LC3 (Abeam, ab48394); β-actin (Sigmα-Aldrich, A5441); TGF-β (Cell Signaling, 3711); TGFβ-RI (Santa Cruz Biotech, sc-398); phospho-Akt (Cell Signaling, 9271); Akt (Cell Signaling, 2967); phospho-mTOR (Cell Signaling, 2971); mTOR (Cell Signaling, 2972); phospho-p70 S6 kinase (Cell Signaling, 9234); p70 S6 kinase (Cell Signaling, 9202); CD31 (BD Biosciences, 550274); Rac1 (Santa Cruz, sc-95); Cdc42 (Santa Cruz, se-8401); p-NFkB (Cell Signaling, 3037); NFkB (Cell Signaling, 3034); p14ARF (Santa Cruz, sc-53639); p16 (Santa Cruz, sc-759); p21 (Santa Cruz, sc-6246); LAMP1 (Santa Cruz, sc-17768); cathepsin B (Santa Cruz, sc-13985).

Cell Culture and Stable Transfection

Human immortalized fibroblasts (hTERT-BJ1) were used to generate the cell lines over-expressing Migration Stimulatory Factor (MSF), SMA, Rac1, and Cdc42. Lentiviral plasmids [EX-NEG-Lv105 (empty vector), Ex- Z4998-Lv105 (human MSF), Ex-D0101-Lv105 (human SMA), Ex-A0247-Lv105 (human Rac1), Ex-X0047-Lv105 (human Cdc42) (obtained from GeneCopoeia, Inc.)] were used to transfect GeneCopoeia 293Ta lentiviral packaging cells using Lenti-Pac™ HIV Expression Packaging Kit following the manufacturer's instructions. After 48 hours, lentivirus containing culture medium was added to human fibroblasts in the presence of 5 μg/ml Polybrene. Infected fibroblasts were selected with puromycin (1.5 μg/ml).

All cell lines used in the following experiments were cultured in Dulbecco's modified Eagle's medium (DMEM), supplemented with 10% fetal bovine serum in a 37° C. humidified atmosphere (5% CO₂), unless otherwise noted.

Immuno-Blot Analysis

For immuno-blotting, cultured cells were harvested in lysis buffer (10 mM Tris, pH 7.5, 150 mM NaCl, 1% Triton X-100, and 60 mM n-octyl-glucoside) or RIPA lysis buffer containing protease inhibitors (Roche, 11836153001) and phosphatase inhibitors (Thermoscientific, 78420). The pooled cells were rotated for 40 min at 4° C., centrifuged at 10.000×g for 15 min at 4° C. and the protein concentration of the supernatant was determined using the BCA reagent (Pierce). Protein samples (30-50 μg total proteins per lane) were then subjected to 12% or 15% SDS-PAGE, and the proteins were then electrophoretically transferred to a nitrocellulose membrane. After blocking for 1 hour at room temperature with TEST (10 mM Tris, pH 8.0, 150 mM NaCl, 0.05% Twccn 20) supplemented with 5% nonfat dry milk and 1% BSA, membranes were incubated for 1 hour at room temperature with primary antibodies and then for 1 hour at RT with specific (HRP)-conjugate secondary antibodies (anti-mouse, 1:6,000 dilution (Pierce) or anti-rabbit, 1:5,000 dilution (BD PharMingen)). HRP activity was visualized by enhanced chemiluminescent substrate (Thermo Scientific) followed by exposure of the membrane to X-ray film.

Migration Assay

The effects of MSF over-expression on fibroblast migration and the effects of MSF-fibroblasts on the migratory potential of MDA-MB-231 cells were measured in vitro using a modified Boyden chamber assay. Briefly, fibroblasts (EV (empty vector) or MSF over-expressing) in 0.5 ml of serum-free Dulbecco's modified Eagle's medium were added to the wells of 8 μm pore uncoated membrane of modified Boyden chambers. The lower chambers contained 10% fetal bovine serum in Dulbecco's modified Eagle's medium to serve as a chemo-attractant. Cells were incubated at 37° C. and allowed to migrate throughout the course of 6 hours. To assess the effect of fibroblast over-expressing MSF protein on the migratory capacity of MDA-MB-231 cells, fibroblasts were seeded in the lower chambers in DMEM supplemented with 10% NuSerum and used as chemo-attractant. MDA-MB-231 cells were added to the wells of 8 μm pore uncoated membrane of modified Boyden chambers and allowed to migrate throughout the course of 4 hours at 37° C. In both cases, cells were removed from the upper surface of the membrane by scrubbing with cotton swabs. Chambers were stained in 0.5% crystal violet diluted in 100% methanol for 30 minutes, rinsed in water and examined under a bright-field microscope. Values for migration were obtained by counting five fields per membrane (X20 objective) and represent the average of three independent experiments.

L-Lactate Assay

Cells (empty vector or MSF-, SMA-, Cdc42- over-expressing fibroblasts) were seeded in quadruplicate (100,000 cells per well) in 12-well plates in 1 ml of complete media. After 18 hours, the media was changed to DMEM containing 2% FBS and incubated under hypoxic conditions (0.5% O₂). SMA and cdc42 over-expressing fibroblasts were also with or without metformin (1 mM). After 48 hours, the media of each well was collected and the concentration of L-lactate was measured using the EnzyChrom™ L-Lactate Assay Kit (ECLC-100, BioAssay Systems, Inc.). After removing the media, cells were tyrpsinized, spun-down and resuspended in 1 mL of media for quantification. Cells were counted in 4-6 fields, using a 40× objective lens and a haemocytometer. Cells were then lysed and the protein concentration was determined using the BCA reagent (Pierce). The amount of L-lactate in the media was normalized to total cell number or to total cell protein content.

Mitochondrial Staining

To evaluate mitochondrial activity, cells were stained with MitoTracker Orange (CMTMRos; M7510, Invitrogen, Inc.). Lyophilized MitoTracker was dissolved in DMSO to generate a 1 mM stock solution that was then diluted into serum-free DMEM at a final concentration of 25 nM. Briefly, control or MSF-, SMA-, Cdc42-over-expressing fibroblasts (90,000 cells per well) were cultured for 48 hours in normoxia or under hypoxic conditions. Then, they were incubated with pre-warmed MitoTracker staining solution for 12 min at 37° C. in the dark. Cells were then washed in PBS Ca²⁺/Mg²⁺, three times and fixed with 2% PFA 30 min a RT. Cell were washed again with PBS Ca²⁺/Mg²⁺, incubated with the nuclear stain DAPI and mounted.

Murine Xenograft Studies

All animals were housed and maintained in a barrier facility at the Kimmel Cancer Center at Thomas Jefferson University under National Institutes of Health (NIH) guidelines. Mice were kept on a 12-hour light/dark cycle with ad libitum access to food and water. Animal protocols used for this study were pre-approved by the Institutional Animal Care and Use Committee (IACUC). Briefly, MDA-MB-231-GFP human breast cancer cells (1×10⁶ cells) were co-injected with control (empty vector) or MSF-, SMA-, Cdc42- over-expressing fibroblasts (3×10⁵ cells) in 100 μl of sterile PBS into the flanks of athymic NCr nude mice (NCRNU; Taconic Farms; 6-8 weeks of age). Mice were then sacrificed at 4-weeks post-injection; tumors were dissected to determine their weight and size using calipers. Tumor volume was calculated using the formula (X²Y)/2, where X and Y are the short and long dimensions, respectively, of the tumor. After the dissection, tumors were fixed with 10% formalin or flash-frozen in liquid nitrogen-cooled isopentane.

Quantitation of Tumor Angiogenesis

Immuno-histochemical staining for CD31 was performed on frozen tumor sections using a 3-step biotin-streptavidin-horseradish peroxidase method. Frozen tissue sections (6 μm) were fixed in 4% paraformaldehyde in PBS for 10 min and washed with PBS. After blocking with 10% rabbit serum, the sections were incubated overnight at 4° C. with rat anti-mouse CD31 antibody (BS Biosciences) at a dilution of 1:200, followed by biotinylated rabbit anti-rat IgG ((Vector Labs, 1:200) antibody and streptavidin-HRP (Daki, 1:1000). Immuno-reactivity was revealed with 3,3′-diaminobenzidine (DAB).

Phalloidin Staining

Cell monolayers were stained with Alexa Fluor 633-phalloidin to examine the structure of filamentous F-actin. Washed cells were fixed with paraformaldehyde 2%, washed again with PBS Ca²⁺/Mg²⁺ and permeabilized for 10 min with TBP buffer (0.1% Triton X-100, 0.2% BSA in PBS Ca²⁺/Mg²⁺. The cells were stained with Phalloidin Staining Solution (diluted 1:40 in PBS 1% BSA) 30 min, at RT in dark conditions. Stained F-actin was visualized using a Zeiss LSM510 meta-confocal system. Images were acquired with a 20× objective.

Statistical Analysis

Statistical significance was examined using the Student's t-test. Values of p≦0.05 were considered significant. Values were expressed as means±SEM.

Acknowledgements

F.S. and her laboratory were supported by grants from the Breast Cancer Alliance (BCA) and the American Cancer Society (ACS). U.E.M. was supported by a Young Investigator Award from the Margaret Q. Landenberger Research Foundation. M.P.L. was supported by grants from the NIH/NCI (R01-CA-080250; R01-CA-098779; R01-CA-120876; R01-AR-055660), and the Susan G. Komen Breast Cancer Foundation. R.G.P. was supported by grants from the NIH/NCI (R01-CA-70896, R01-CA-75503, R01-CA-86072, and R01-CA-107382) and the Dr. Ralph and Marian C. Falk Medical Research Trust. The Kimmel Cancer Center was supported by the NIH/NCI Cancer Center Core grant P30-CA-56036 (to R.G.P.). Funds were also contributed by the Margaret Q. Landenberger Research Foundation (to M.P.L. and U.E.M.-O.). This project is funded, in part, under a grant with the Pennsylvania Department of Health (to M.P.L. and F.S.). The Department specifically disclaims responsibility for any analyses, interpretations or conclusions. This work was also supported, in part, by a Centre grant in Manchester from Breakthrough Breast Cancer in the U.K. (to A.H.) and an Advanced ERC Grant from the European Research Council.

### REFERENCES

1. Petersen O W, Nielsen H L, Gudjonsson T, Villadsen R, Rank F, Niebuhr E, Bissell M J, Ronnov-Jessen L. Epithelial to mesenchymal transition in human breast cancer can provide a nonmalignant stroma. Am J Pathol 2003; 162:391-402.
2. Ronnov-Jessen L, Petersen O W, Bissell M J. Cellular changes involved in conversion of normal to malignant breast: importance of the stromal reaction. Physiol Rev 1996; 76:69-125.
3. Sieweke M H, Bissell M J. The tumor-promoting effect of wounding: a possible role for TGF-beta-induced stromal alterations. Crit Rev Oncog 1994; 5:297-311.
4. Sieweke M H, Thompson N L, Sporn M B, Bissell M J. Mediation of wound-related Rous sarcoma virus tumorigenesis by TGF-beta. Science 1990; 248:1656-60.
5. Direkze N C, Hodivala-Dilke K, Jeffery R, Hunt T, Poulsom R, Oukrif D, Alison M R, Wright N A. Bone marrow contribution to tumor-associated myofibroblasts and fibroblasts. Cancer Res 2004; 64:8492-5.
6. Santos A M, Jung J, Aziz N, Kissil J L, Pure E. Targeting fibroblast activation protein inhibits tumor stromagenesis and growth in mice. J Clin Invest 2009; 119:3613-25.
7. Zeisberg E M, Potenta S, Xie L, Zeisberg M, Kalluri R. Discovery of endothelial to mesenchymal transition as a source for carcinoma-associated fibroblasts. Cancer Res 2007; 67:10123-8.
8. Martinez-Outschoorn U E, Sotgia F, Lisanti M P. Power surge: supporting cells “fuel” cancer cell mitochondria. Cell Metab 2012; 15:4-5.
9. Sotgia F, Martinez-Outschoorn U E, Howell A, Pestell R G, Pavlides S, Lisanti M P. Caveolin-1 and cancer metabolism in the tumor microenvironment: markers, models, and mechanisms. Annu Rev Pathol 2012; 7:423-67.
10. Martinez-Outschoorn U E, Pavlides S, Howell A, Pestell R G, Tanowitz H B, Sotgia F, Lisanti M P. Stromal-epithelial metabolic coupling in cancer: integrating autophagy and metabolism in the tumor microenvironment. Int J Biochem Cell Biol 2011; 43:1045-51.
11. Martinez-Outschoorn U E, Pavlides S, Sotgia F, Lisanti M P. Mitochondrial biogenesis drives tumor cell proliferation. Am J Pathol 2011; 178:1949-52.
12. Whitaker-Menezes D, Martinez-Outschoorn U E, Flomenberg N, Birbe R C, Witkiewicz A K, Howell A, Pavlides S, Tsirigos A, Ertel A, Pestell R G, Broda P, Minetti C, Lisanti M P, Sotgia F. Hyperactivation of oxidative mitochondrial metabolism in epithelial cancer cells in situ: visualizing the therapeutic effects of metformin in tumor tissue. Cell Cycle 2011; 10:4047-64.
13. Whitaker-Menezes D, Martinez-Outschoorn U E, Lin Z, Ertel A, Flomenberg N, Witkicwicz A K, Birbe R C, Howell A, Pavlides S, Gandara R, Pestell R G, Sotgia F, Philp N J, Lisanti M P. Evidence for a stromal-epithelial “lactate shuttle” in human tumors: MCT4 is a marker of oxidative stress in cancer-associated fibroblasts. Cell Cycle 2011; 10:1772-83.
14. Pavlides S, Vera I, Gandara R, Sneddon S, Pestell R G, Mercier I, Martinez-Outschoorn U E, Whitaker-Menezes D, Howell A, Sotgia F, Lisanti M P. Warburg meets autophagy: cancer-associated fibroblasts accelerate tumor growth and metastasis via oxidative stress, mitophagy, and aerobic glycolysis. Antioxid Redox Signal 2012; 16:1264-84.
15. Pavlides S, Whitaker-Menezes D, Castello-Cros R, Flomenberg N, Witkiewicz A K, Frank P G, Casimiro M C, Wang C, Fortina P, Addya S, Pestell R G, Martinez-Outschoorn U E, Sotgia F, Lisanti M P. The reverse Warburg effect: aerobic glycolysis in cancer associated fibroblasts and the tumor stroma. Cell Cycle 2009; 8:3984-4001.
16. Casey T M, Eneman J, Crocker A, White J, Tessitore J, Stanley M, Harlow S, Bunn J Y, Weaver D, Muss H, Plaut K. Cancer associated fibroblasts stimulated by transforming growth factor betal (TGF-beta 1) increase invasion rate of tumor cells: a population study. Breast Cancer Res Treat 2008; 110:39-49.
17. Desmouliere A, Geinoz A, Gabbiani F, Gabbiani G. Transforming growth factor-beta 1 induces alpha-smooth muscle actin expression in granulation tissue myofibroblasts and in quiescent and growing cultured fibroblasts. J Cell Biol 1993; 122:103-11.
18. Kojima Y, Acar A, Eaton E N, Mellody K T, Scheel C, Ben-Porath I, Onder T T, Wang Z C, Richardson A L, Weinberg R A, Orimo A. Autocrine TGF-beta and stromal cell-derived factor-1 (SDF-1) signaling drives the evolution of tumor-promoting mammary stromal myofibroblasts. Proc Nati Acad Sci U S A 2010; 107:20009-14.
19. Martinez-Outschoorn U E, Pavlides S, Whitaker-Menczes D, Daumer K M, Milliman J N, Chiavarina B, Migneco G, Witkiewicz A K, Martinez-Cantarin M P, Flomenberg N, Howell A, Pestell R G, Lisanti M P, Sotgia F. Tumor cells induce the cancer associated fibroblast phenotype via caveolin-1 degradation: Implications for breast cancer and DCIS therapy with autophagy inhibitors. Cell Cycle 2010; 9.
20. Mishra P J, Humeniuk R, Medina D J, Alexe G, Mesirov J P, Ganesan S, Glod J W, Banerjee D. Carcinoma-associated fibroblast-like differentiation of human mesenchymal stem cells. Cancer Res 2008; 68:4331-9.
21. Ronnov-Jessen L, Petersen O W, Induction of alpha-smooth muscle actin by transforming growth factor-beta 1 in quiescent human breast gland fibroblasts. Implications for myofibroblast generation in breast neoplasia. Lab Invest 1993; 68:696-707.
22. Surowiak P, Murawa D, Materna V, Macicjczyk A, Pudelko M, Ciesla S, Breborowicz J, Murawa P, Zabel M, Dietel M, Lage H. Occurence of stromal myofibroblasts in the invasive ductal breast cancer tissue is an unfavourable prognostic factor. Anticancer Res 2007; 27:2917-24.
23. Toullee A, Gerald D, Despouy G, Bourachot B, Cardon M, Lefort S, Richardson M, Rigaill G, Parrini M C, Lucchesi C, Bellanger D, Stern M H, Dubois T, Sastre-Garau X, Delattre O, Vincent-Salomon A, Mechta-Grigoriou F. Oxidative stress promotes myofibroblast differentiation and tumour spreading. EMBO Mol Med 2010; 2:211-30.
24. Tsujino T, Seshimo I, Yamamoto H, Ngan CY, Ezumi K, Takcmasa I, Ikeda M, Sekimoto M, Matsuura N, Monden M. Stromal myofibroblasts predict disease recurrence for colorectal cancer. Clin Cancer Res 2007; 13:2082-90.
25. Vozenin-Brotons M C, Sivan V, Gault N, Renard C, Geffrotin C, Delanian S, Lefaix J L, Martin M. Antifibrotic action of Cu/Zn SOD is mediated by TGF-beta 1 repression and phenotypic reversion of myofibroblasts. Free Radic Biol Med 2001; 30:30-42.
26. Waghray M, Cui Z, Horowitz J C, Subramanian I M, Martinez F J, Toews G B, Thannickal V J. Hydrogen peroxide is a diffusible paracrine signal for the induction of epithelial cell death by activated myofibroblasts. FASEB J 2005; 19:854-6.
27. Schor S L, Haggie J A, Durning P, Howell A, Smith L, Sellwood R A, Crowther D. Occurrence of a fetal fibroblast phenotype in familial breast cancer. Int J Cancer 1986; 37:831-6.
28. Schor S L, Schor A M, Howell A, Crowther D. Hypothesis: persistent expression of fetal phenotypic characteristics by fibroblasts is associated with an increased susceptibility to neoplastic disease. Exp Cell Biol 1987; 55:11-7.
29. Haggie J A, Sellwood R A, Howell A, Birch J M, Schor SL. Fibroblasts from relatives of patients with hereditary breast cancer show fetal-like behaviour in vitro. Lancet 1987; 1:1455-7.
30. Colletta A A, Wakefield L M, Howell F V, van Roozendaal K E, Danielpour D, Ebbs S R, Sporn M B, Baum M. Anti-oestrogens induce the secretion of active transforming growth factor beta from human fetal fibroblasts. Br J Cancer 1990; 62:405-9.
31. Schor S L, Grey A M, Picardo M, Schor A M, Howell A, Ellis I, Rushton G. Heterogeneity amongst fibroblasts in the production of migration stimulating factor (MSF): implications for cancer pathogenesis. Exs 1991; 59:127-46.
32. Picardo M, Schor S L, Grey A M, Howell A, Laidlaw I, Redford J, Schor A M. Migration stimulating activity in serum of breast cancer patients. Lancet 1991; 337:130-3.
33. Schor S L, Grey A M, Ellis I, Schor A M, Howell A, Sloan P, Murphy R. Fetal-like fibroblasts: their production of migration-stimulating factor and role in tumor progression. Cancer Treat Res 1994; 71:277-98.
34. Schor A M, Rushton G, Ferguson J E, Howell A, Redford J, Schor S L. Phenotypic heterogeneity in breast fibroblasts: functional anomaly in fibroblasts from histologically normal tissue adjacent to carcinoma. Int J Cancer 1994; 59:25-32.
35. Schor S L, Ellis I R, Jones S J, Baillie R, Seneviratne K, Clausen J, Motegi K, Vojtesek B, Kankova K, Furrie E, Sales M J, Schor A M, Kay R A. Migration-stimulating factor: a genetically truncated onco-fetal fibronectin isoform expressed by carcinoma and tumor-associated stromal cells. Cancer Res 2003; 63:8827-36.
36. Kay R A, Ellis I R, Jones S J, Perrier 5, Florence M M, Schor A M, Schor S L. The expression of migration stimulating factor, a potent oncofetal cytokine, is uniquely controlled by 3′-untranslated region-dependent nuclear sequestration of its precursor messenger RNA. Cancer Res 2005; 65:10742-9.
37. Schor S L, Schor A M, Rushton G. Fibroblasts from cancer patients display a mixture of both foetal and adult-like phenotypic characteristics. J Cell Sci 1988; 90 (Pt 3):401-7.
38. Schor S L, Schor A M, Grey A M, Rushton G. Foetal and cancer patient fibroblasts produce an autocrine migration-stimulating factor not made by normal adult cells. J Cell Sci 1988; 90 (Pt 3):391-9.
39. Schor S L, Schor A M. Foetal-to-adult transitions in fibroblast phenotype: their possible relevance to the pathogenesis of cancer. J Cell Sci Suppl 1987; 8:165-80.
40. Grey A M, Schor A M, Rushton G, Ellis I, Schor S L. Purification of the migration stimulating factor produced by fetal and breast cancer patient fibroblasts. Proc Natl Acad Sci U S A 1989; 86:2438-42.
41. Schor S L, Schor A M, Grey A M, Chen J, Rushton G, Grant M E, Ellis I. Mechanism of action of the migration stimulating factor produced by fetal and cancer patient fibroblasts: effect on hyaluronic and synthesis. In Vitro Cell Dev Biol 1989; 25:737-46.
42. Schor S L, Schor A M. Characterization of migration-stimulating factor (MSF): evidence for its role in cancer pathogenesis. Cancer Invest 1990; 8:665-7.
43. Ellis I, Grey A M, Schor A M, Schor S L. Antagonistic effects of TGF-beta 1 and MSF on fibroblast migration and hyaluronic acid synthesis. Possible implications for dermal wound healing. J Cell Sci 1992; 102 (Pt 3):447-56.
44, Schor S L, Grey A M, Ellis I, Schor A M, Coles B, Murphy R. Migration stimulating factor (MSF): its structure, mode of action and possible function in health and disease. Symp Soc Exp Biol 1993; 47:235-51.
45. Schor S L. Fibroblast subpopulations as accelerators of tumor progression: the role of migration stimulating factor. Exs 1995; 74:273-96.
46. Schor S L, Schor A M. Phenotypic and genetic alterations in mammary stroma: implications for tumour progression. Breast Cancer Res 2001; 3:373-9.
47. Levy L, Hill C S. Alterations in components of the TGF-beta superfamily signaling pathways in human cancer. Cytokine Growth Factor Rev 2006; 17:41-58.
48. Lohr M, Schmidt C, Ringel J, Kluth M, Muller P, Nizze H, Jesnowski R. Transforming growth factor-betal induces desmoplasia in an experimental model of human pancreatic carcinoma. Cancer Res 2001; 61:550-5.
49. Massague J. TGFbeta in Cancer. Cell 2008; 134:215-30.
50. Massague J. TGF-beta signaling in development and disease. FEBS Lett 2012; 586:1833.
51. Racker E, Resnick R J, Feldman R. Glycolysis and methylaminoisobutyrate uptake in rat-1 cells transfected with ras or myc oncogenes. Proc Natl Acad Sci U S A 1985; 82:3535-8.
52. Nowak G, Schnellmann R G. Autocrine production and TGF-beta 1-mediated effects on metabolism and viability in renal cells. Am J Physiol 1996; 271:F689-97.
53. Monypenny J, Zicha D, Higashida C, Oceguera-Yanez F, Narumiya S, Watanabe N. Cdc42 and Rac family GTPases regulate mode and speed but not direction of primary fibroblast migration during platelet-derived growth factor-dependent chemotaxis. Mol Cell Biol 2009; 29:2730-47.
54. Heasman S J, Ridley A J. Mammalian Rho GTPases: new insights into their functions from in vivo studies. Nat Rev Mol Cell Biol 2008; 9:690-701.

### FIGURE LEGENDS

FIG. 1. MSF Over-Expression Confers the Cancer-Associated Fibroblast Phenotype, Characterized by the Expression of Myo-Fibroblast Markers and Activated TGF-β Signaling.

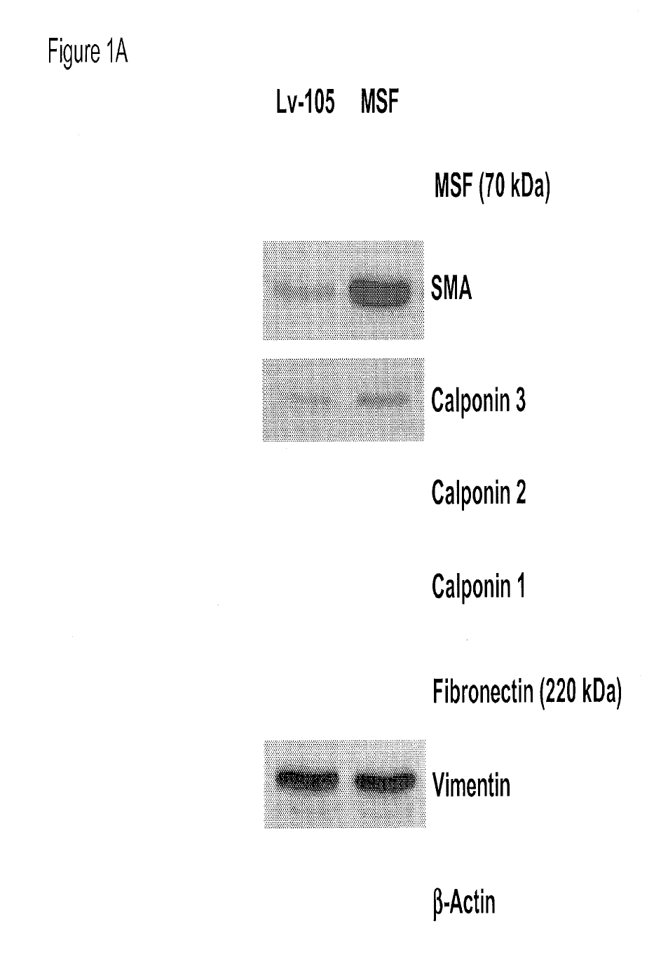

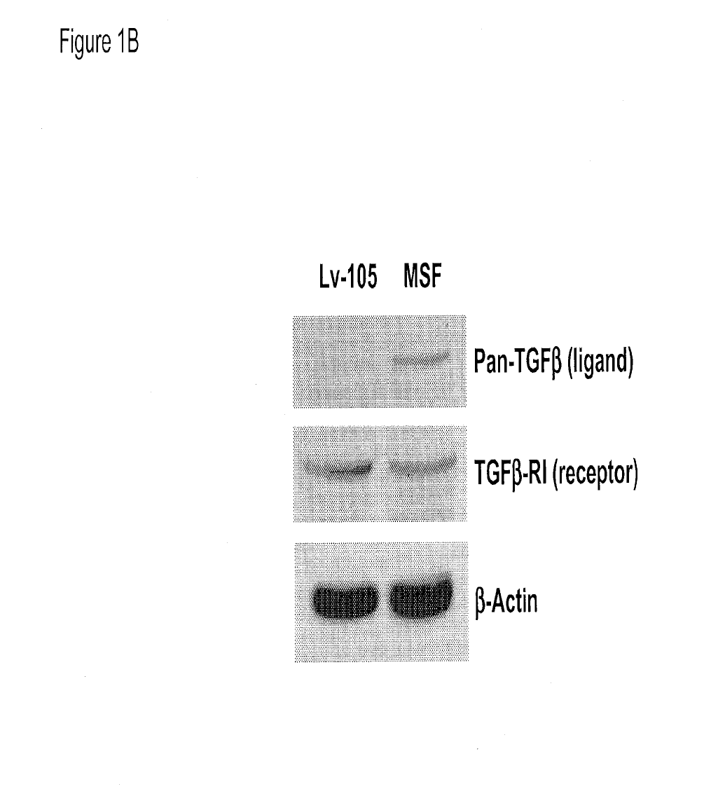

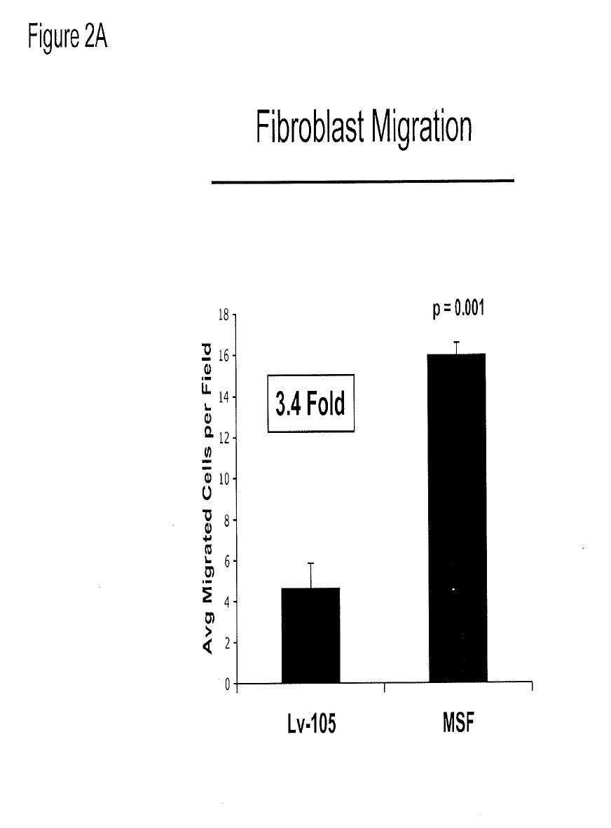

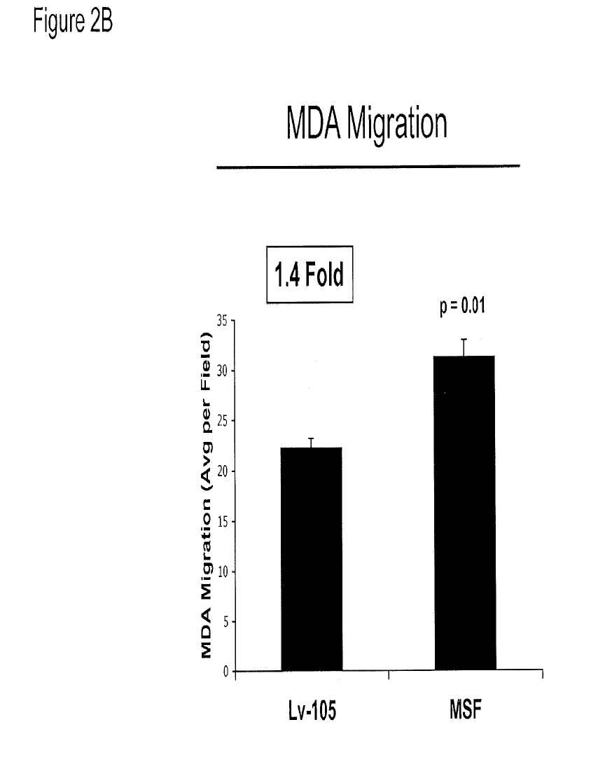

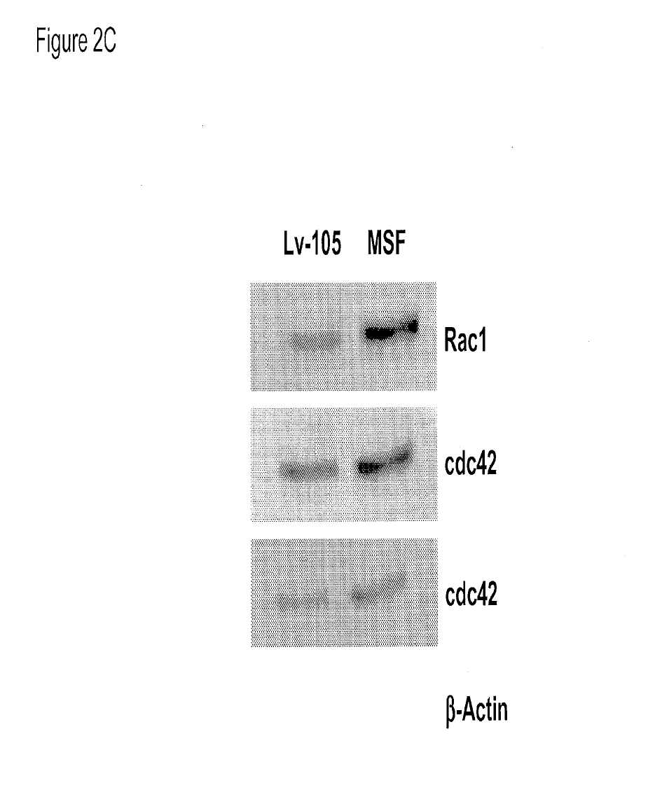

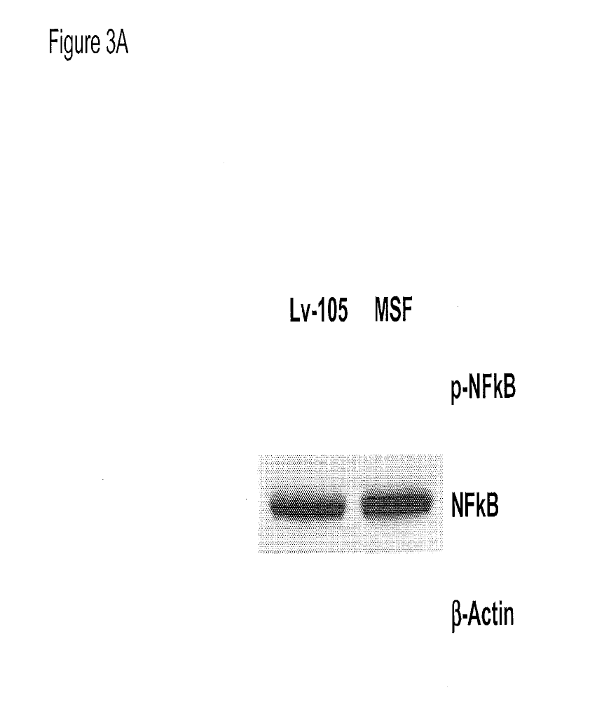

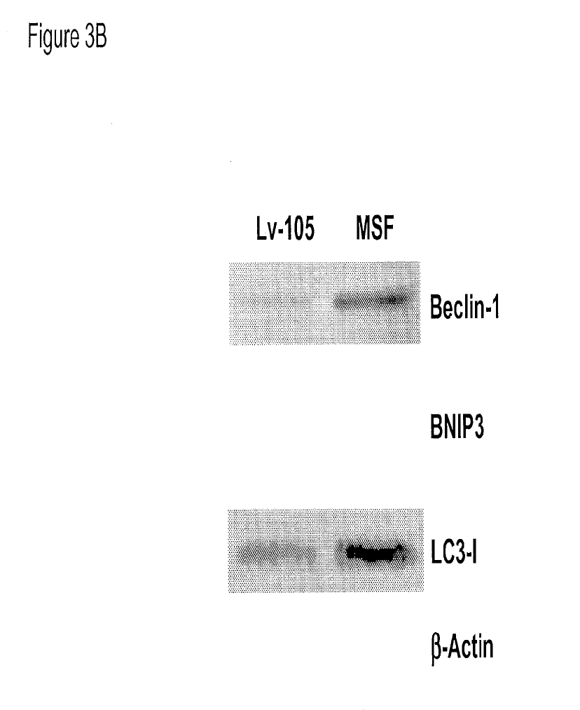

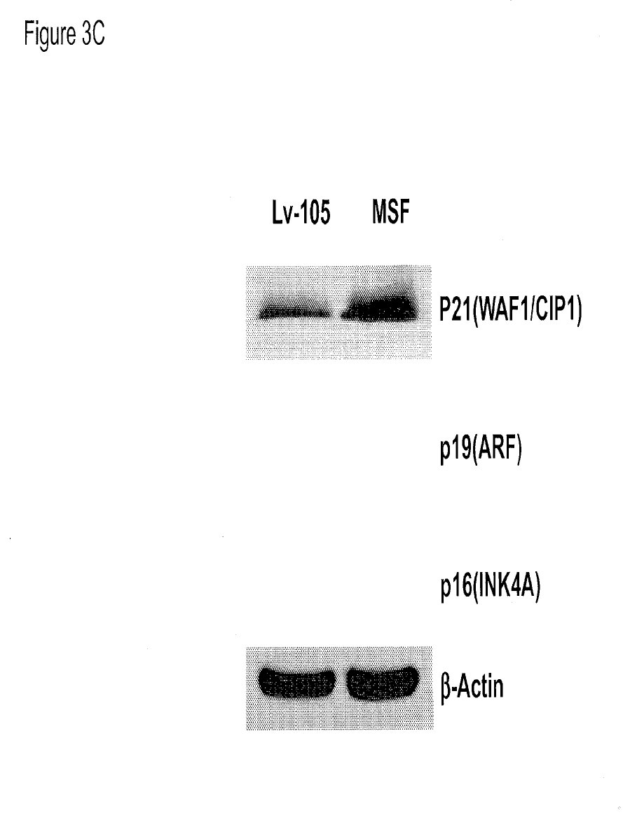

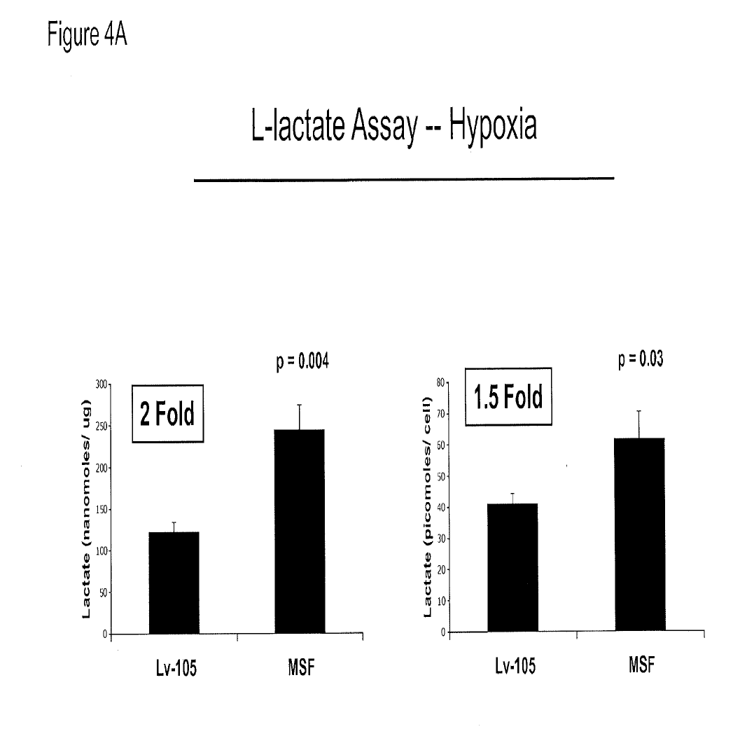

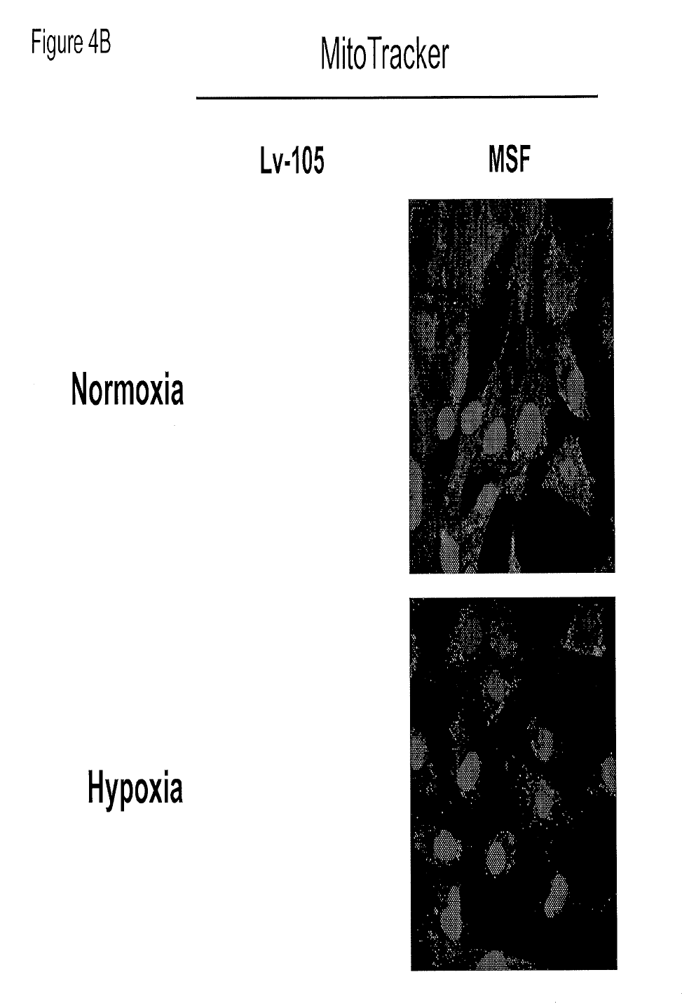

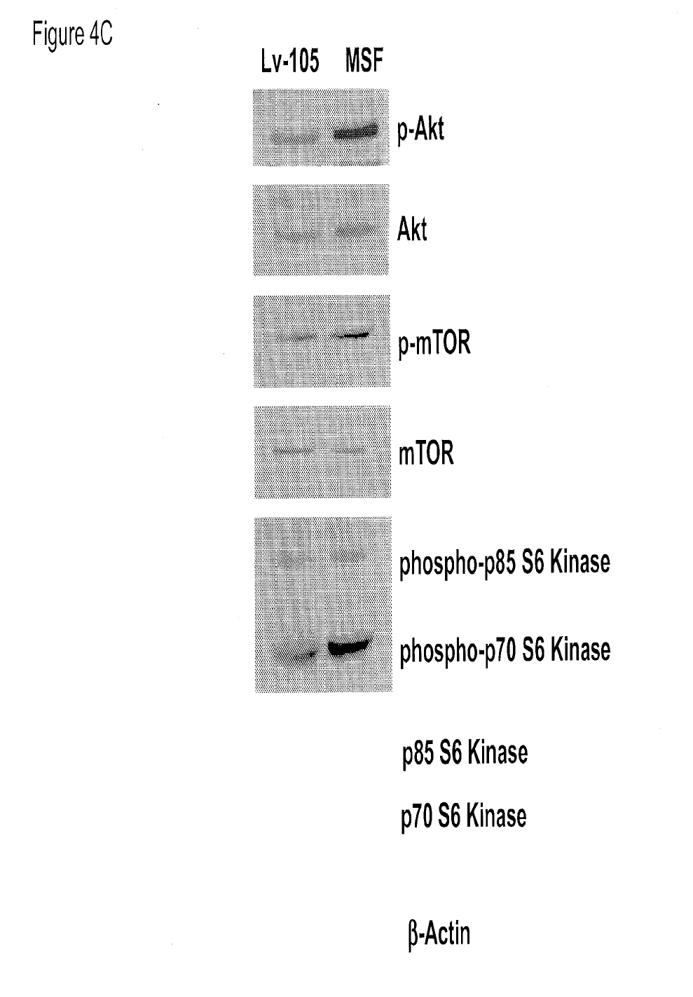

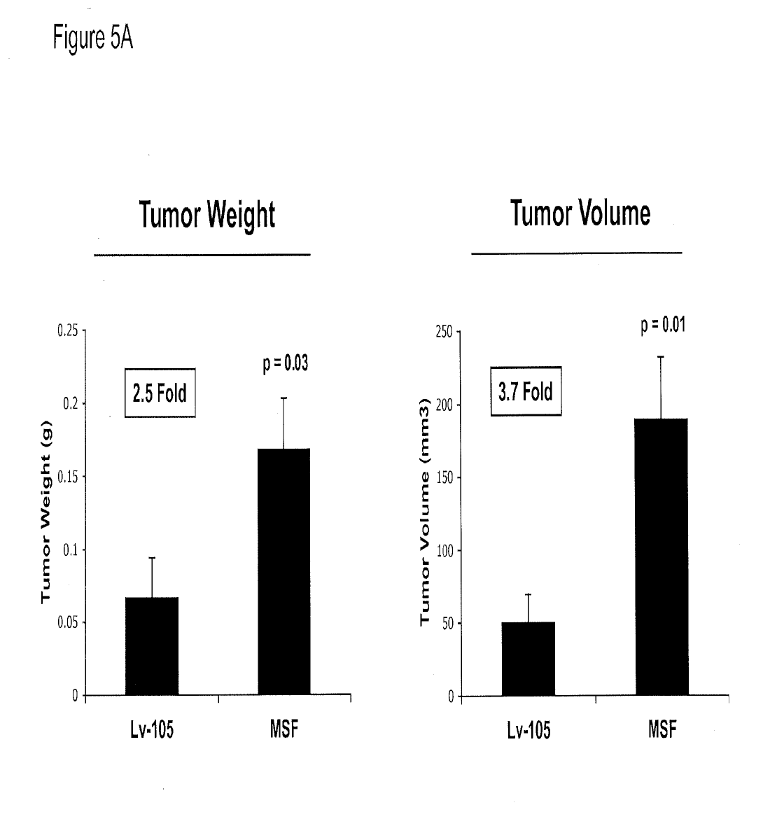

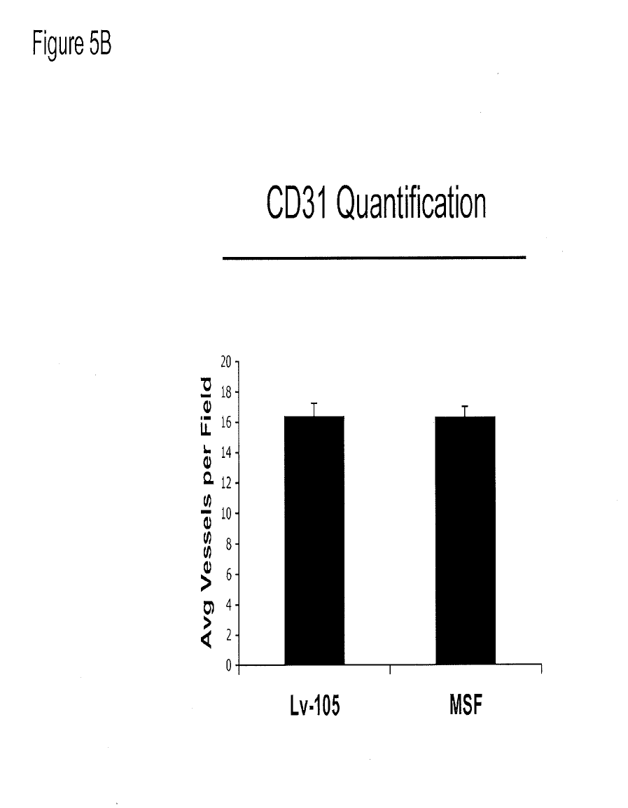

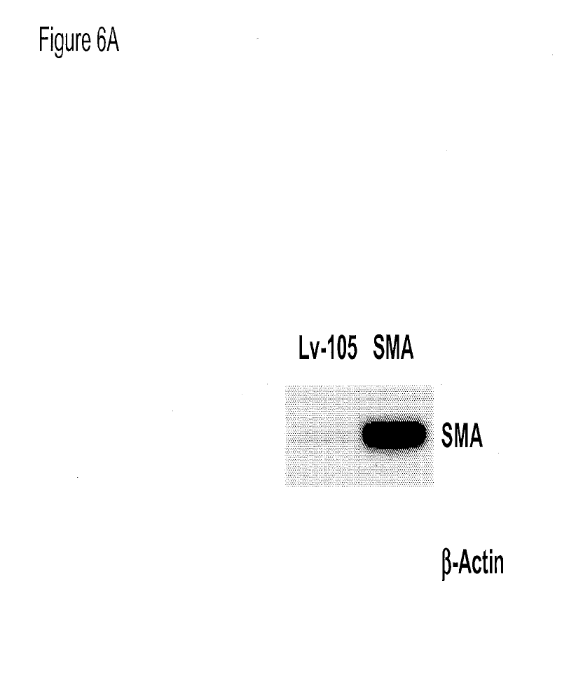

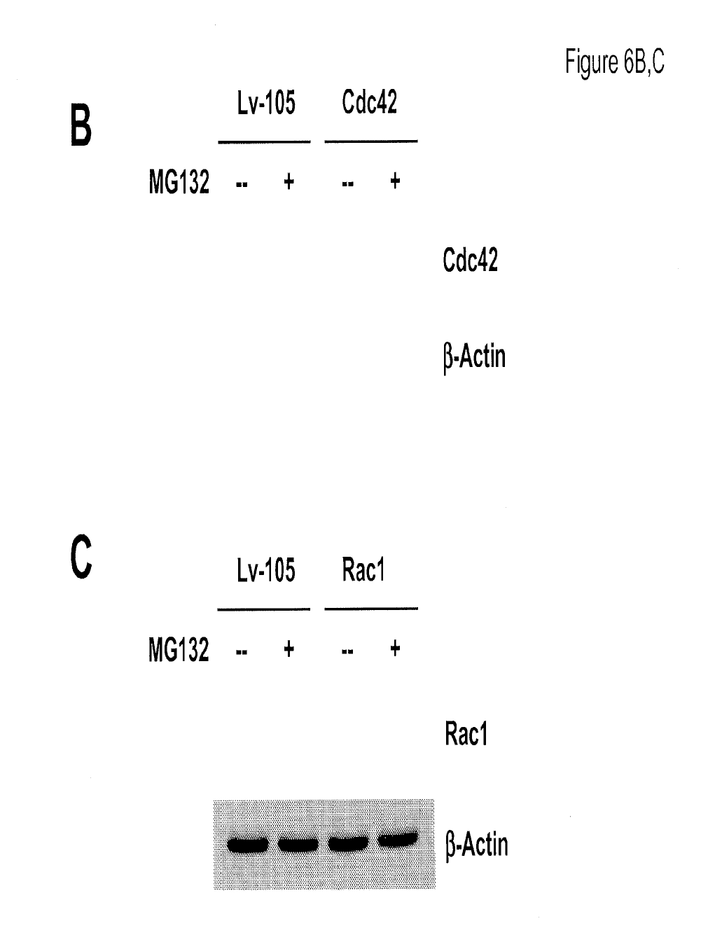

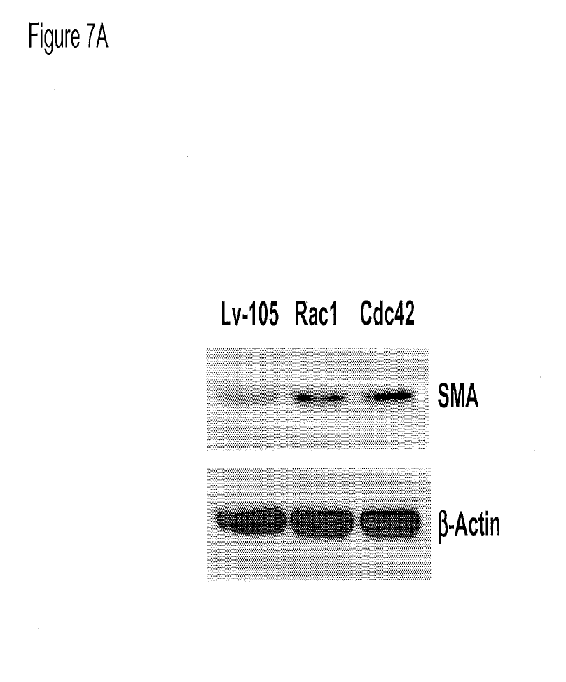

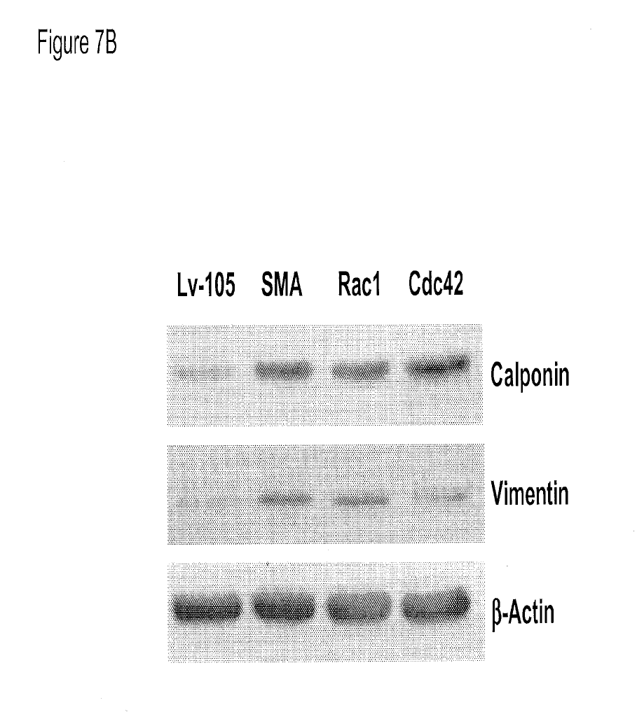

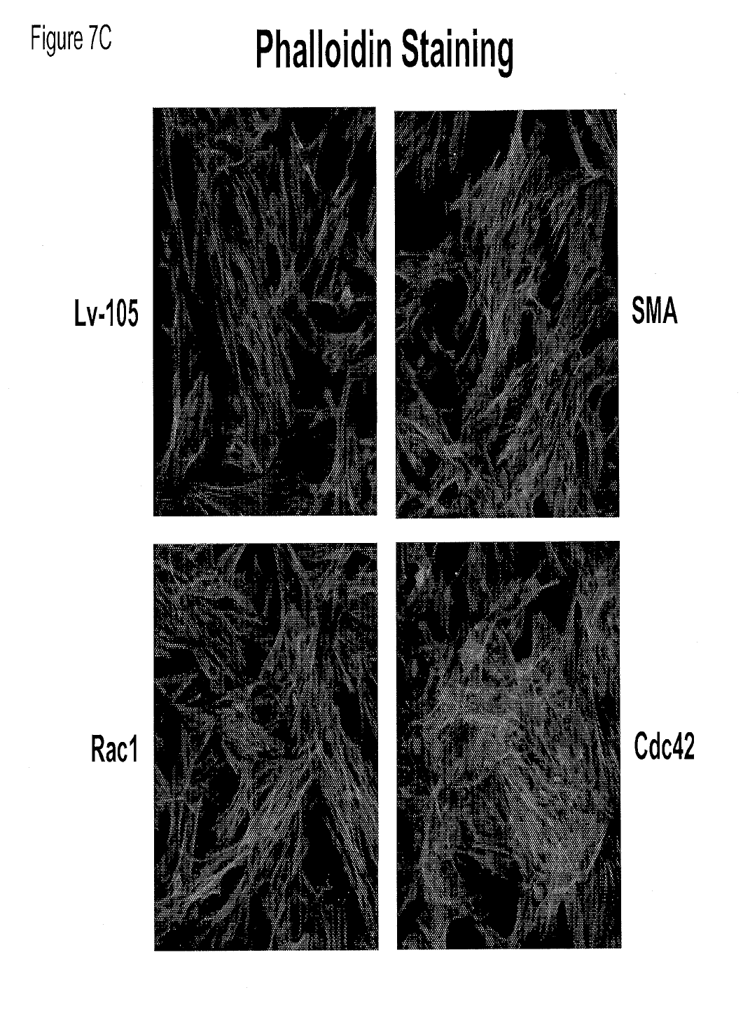

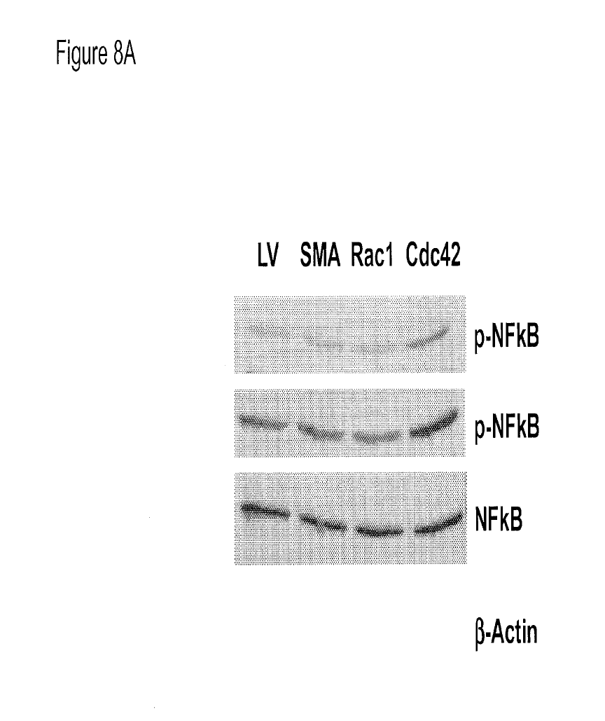

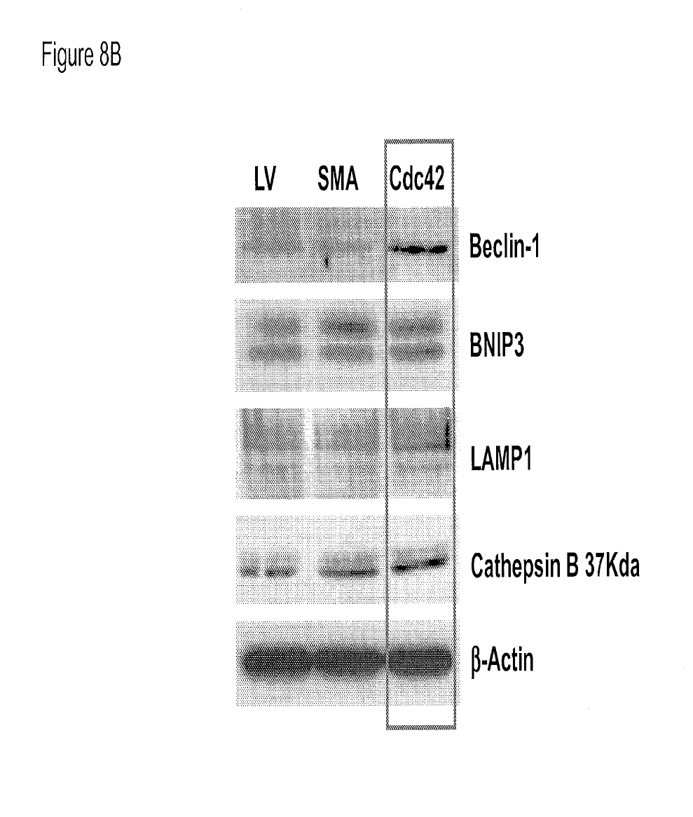

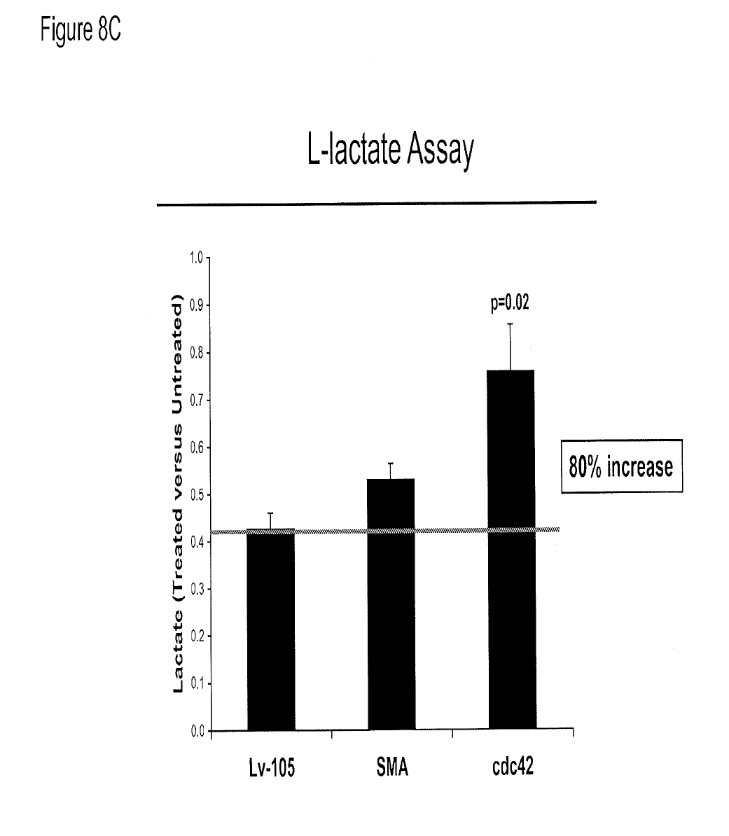

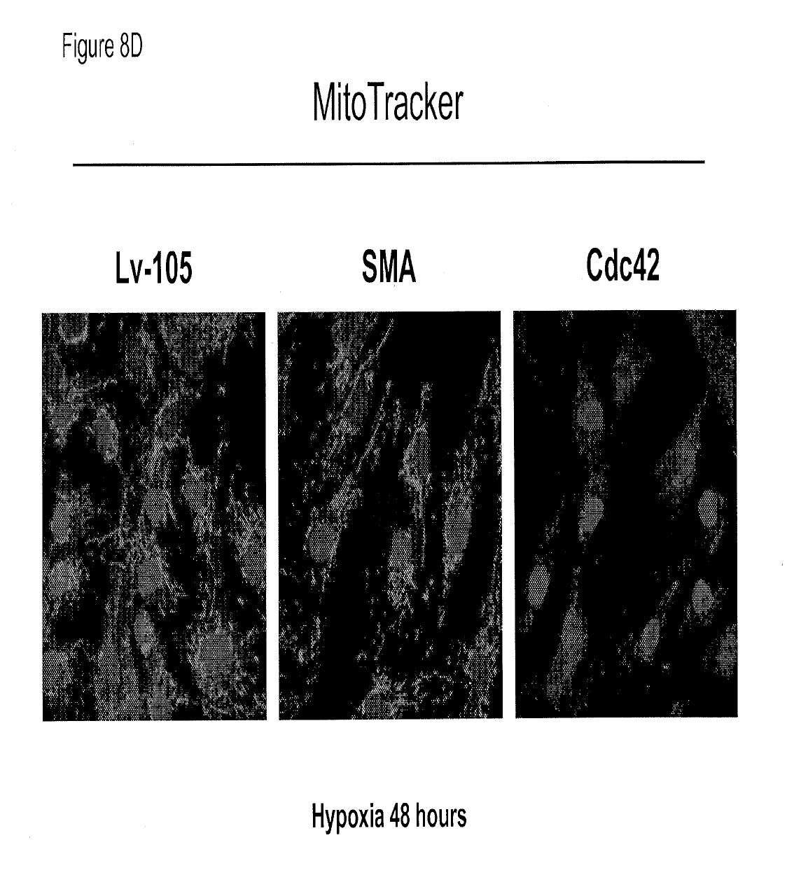

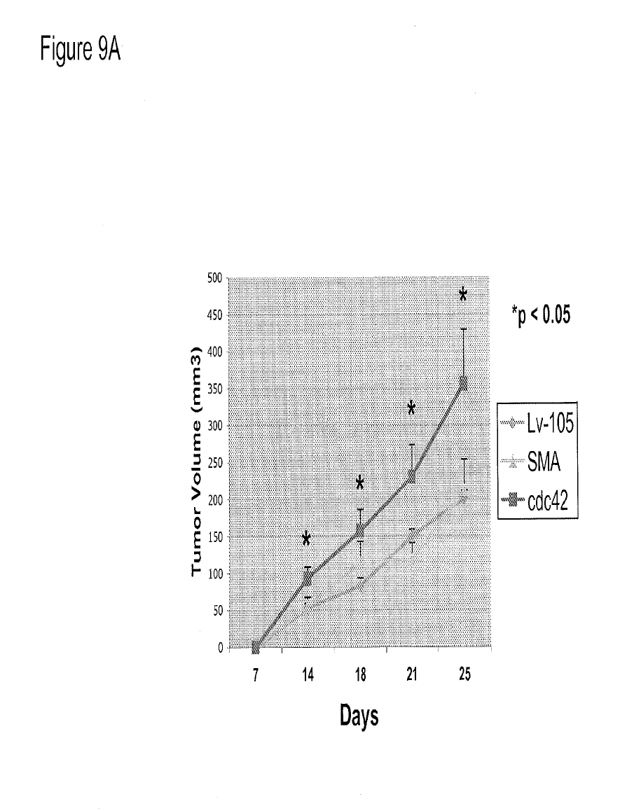

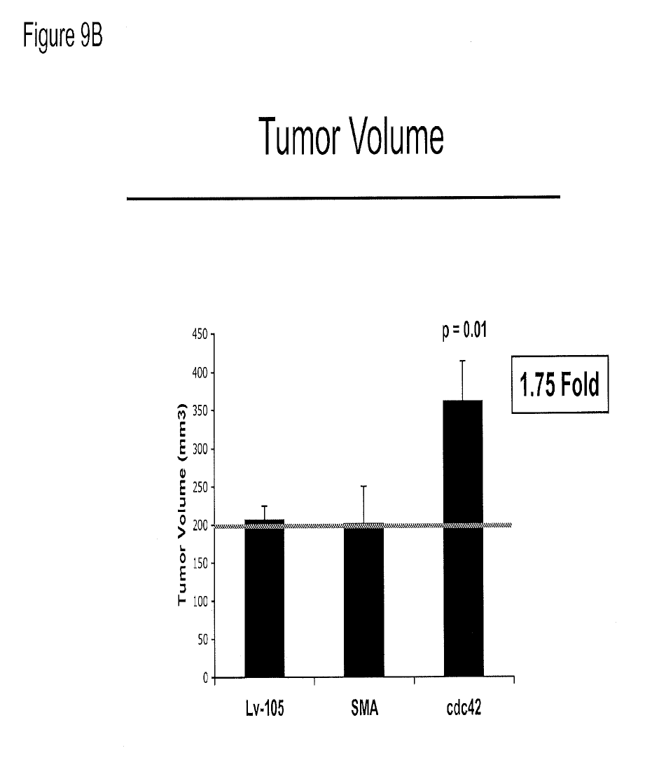

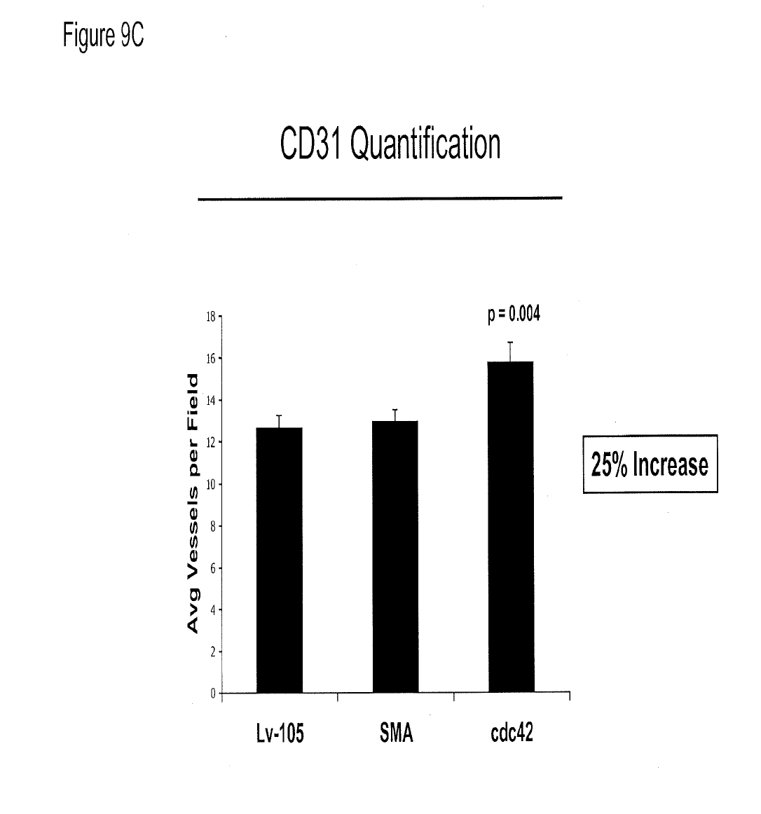

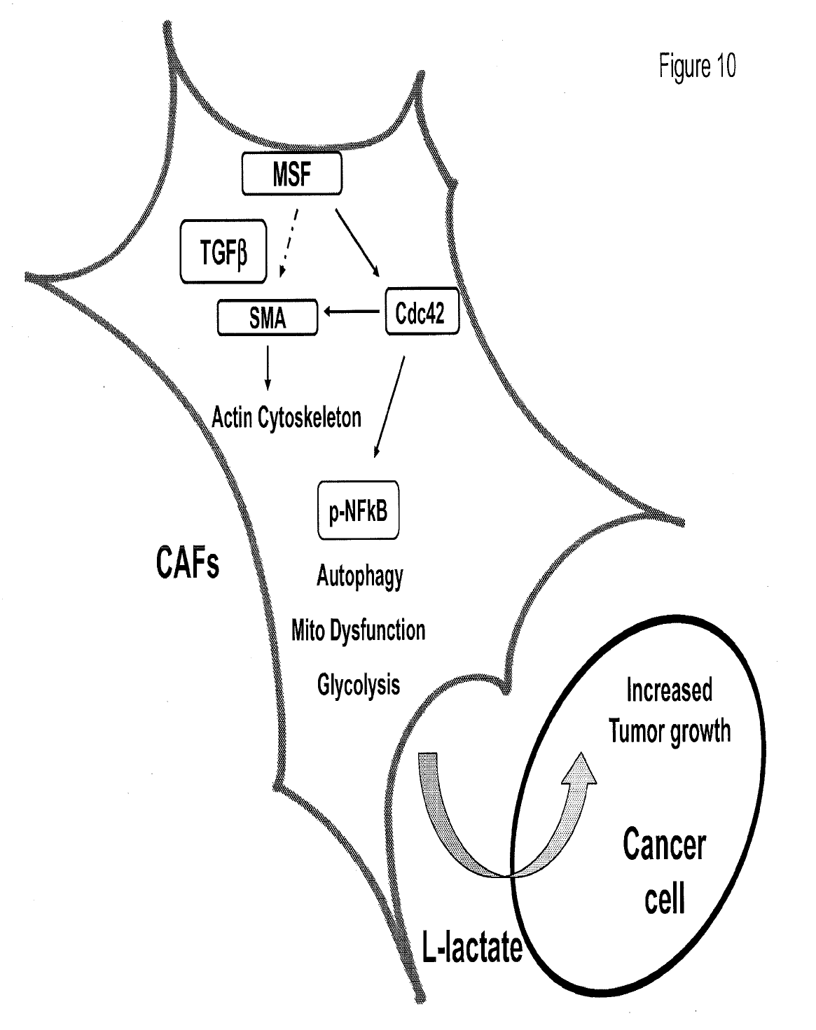

(A) To assess the functional role of MSF stromal expression in tumor growth, we stably over-expressed the MSF protein in immortalized human fibroblast cell line (hTERT-BJ1 cells). Successful over-expression of the MSF protein was verified by immuno-blot analysis. Empty vector control (Lv-105) or MSF fibroblasts were also subjected to immuno-blot analysis with different myofibroblast marker proteins. Equal protein loading was assessed by immunoblotting with β-actin. Note that MSF over-expression strongly induces the expression of myofibroblast markers such as α-SMA, calponin 1 and 3 and fibronectin, while no increases in vimentin protein expression were observed in the fibroblasts.

(B) MSF over-expression induces the activation of TGF-β signaling. Note that immuno-blot analysis of control and MSF fibroblasts reveals that TGF-β ligand protein expression is significantly increased in MSF fibroblasts. Conversely, MSF fibroblasts are characterized by a reduction in the protein expression of TGFβ-RI (type I receptor), as compared with control fibroblasts.

FIG. 2. MSF Stimulates the Migration of Fibroblasts and Acts as Chemo-Attractant, Stimulating Cancer Cell Migration.

(A) The migration of fibroblasts across an 8 μm pore uncoated membrane was assessed after a 6-hour period, using a modified “Boyden Chamber” assay, employing Transwell cell culture inserts. Interestingly, the motility of fibroblasts over-expressing MSF was increased by ˜3.4-fold, as compared with fibroblasts transfected with the empty-vector (Lv-105); p=001 relative to control migration (Student's t-test).

(B) MSF over-expression stimulates cancer cell migration. To assess whether MSF fibroblasts can function as chemo-attractants, MDA-MB-231 cells were placed in the upper chambers and fibroblasts (empty vector (Lv-105) fibroblasts or MSF fibroblasts) were seeded into the lower chambers. Note that MSF fibroblasts promote cancer cell migration by ˜1.4-fold. p=0.01, control versus MSF fibroblasts (Student's t-test).

(C) MSF is sufficient to increase the expression of the two small GTPases, namely Rac1 and Cdc42. Cells (empty vector (Lv-105) fibroblasts or MSF fibroblasts) were subjected to immuno-blot analysis using antibodies directed against the two small GTPases. Note that MSF increases the expression of proteins, Rac1 and Cdc42.

FIG. 3. MSF Over-Expression in Fibroblasts Promotes the Activation of NFkB, Increases Autophagy, and Induces CDK Inhibitors.

(A) Since the human Rac-1 and Cdc42 proteins efficiently induce the transcriptional activity of nuclear factor kappaB (NF-kappaB), we next evaluated if MSF is able to induce not only the upregulation of the two small GTPases but also the activation of NFkB. Note that immuno-blot analysis of control or MSF fibroblasts revealed that the levels of p-NFkB are significantly increased in MSF fibroblasts, as compared with control fibroblasts.

(B) NFkB can induce autophagy, so we verified if activation of NFkB by MSF overexpressed fibroblasts is sufficient to induce autophagy. Cells were lysed and subjected to immunoblot analysis using antibodies directed against a panel of autophagy markers. β-actin was used as equal loading control. Note that MSF increases the expression of several key autophagy markers, namely Beclin-1, BNIP3, and LC3-1.

(C) To determine if MSF expression is also associated with senescence, we investigated whether known CDK inhibitors are upregulated. Immuno-blot analysis shows that p21 (WAF1/CIP1), p19(ARF), and p16(INK4A) are all upregulated, consistent with cell cycle arrest and/or the onset of senescence.

FIG. 4. MSF Over-Expression in Fibroblasts Induces a Shift Towards Glycolytic Metabolism.

(A) Under hypoxic conditions, MSF fibroblasts secrete elevated levels of L-lactate, consistent with increased aerobic glycolysis. As shown in the figure, MSF fibroblasts secrete increased levels of L-lactate (˜2-fold, p=0.004; normalized for protein content; ˜1.5-fold, p=0.03; normalized for cell number), relative to control fibroblasts processed in parallel. This suggests that MSF over-expressing fibroblasts could provide essential energetic support for adjacent tumor epithelial cells, via the secretion of L-lactate, that is a critical mitochondrial fuel for the tumor growth.

(B) MSF over-expression induces a decrease in mitochondrial activity, as visualized using MitoTracker staining. Note that MSF decreases mitochondrial activity, both under normoxic condition (Upper panels) and under hypoxic condition (Lower panels). MitoTracker (red); nuclei/DAPI (blue). Original magnification, 60×.

(C) MSF activates the Akt pathway. Control or MSF fibroblasts were subjected to immuno-blot analysis with antibodies to phospho-Akt, phospho-mTOR and phospho-p70 S6 kinase. The phospho-Akt, phospho-mTOR and phospho-p70 S6 kinase immunoblots were then reprobed with Akt, mTOR and p70 S6 kinase antibodies, respectively. Notc that MSF over-expression is associated with Akt pathway activation. This reaction is probably an anti-apoptotic response, to protect the fibroblasts from autophagy-induced cell death.

FIG. 5. MSF Over-Expression in Fibroblasts Dramatically Promotes Breast Cancer Tumor Growth, without Affecting Tumor Angiogenesis.

(A) Control or MSF fibroblasts were co-injected with MDA-MB-231 breast cancer cells into the flanks of nude mice. After 4 weeks post-injection, the resulting tumors were harvested. Interestingly, relative to control fibroblasts (Lv-105), MSF fibroblasts increased tumor weight by ˜2.5-fold (p=0.03) and tumor volume by ˜4-fold (p=0.01). n=10 tumors per experimental group.

(B) MSF over-expressing fibroblasts do not increase tumor angiogenesis. Frozen tumor sections were immuno-stained with anti-CD31 antibodies to quantify vessel density. As shown in the figure, note that the tumor promoting effect of MSF fibroblasts are independent of angiogenesis, as no significant increases in vessel density were observed.

FIG. 6. Recombinant Expression of SMA, Rac1, And Cdc42 in Immortalized Fibroblasts.

To assess the role of SMA, Rac1, and Cdc42 in the tumor microenvironment, we stably over-expressed SMA, Rac1, and Cdc42 in the immortalized human fibroblast cell line (hTERT-BJ1 cells). Successful protein over-expression of SMA, Rac1, and Cdc42 was validated by immuno-blot analysis (A, B, C). In order to better visualize Cdc42 and Rac1 over-expression, we treated fibroblasts with a protease inhibitor (MG132; 10 μm for 16 hours).

FIG. 7. Over-Expression of SMA, Rac1, and Cdc42 Confers the Myo-Fibroblast Phenotype.

(A) Rac1- and Cdc42-over-expressing fibroblasts display the up-regulation of SMA protein expression.

(B) Rac1- and Cdc42-induce the expression of other myofibroblast markers, namely calponin and vimentin.

(C) Rac1 and Cdc42 regulate the organization of the actin cytoskeleton, inducing a reorganization of F-actin, as suggested by the increase of actin stress fibers. Equal cell numbers were plated on glass cover sides, and after 24 hours, the cells were fixed and actin filaments and nuclei were stained with Phalloidin (red) and DAPI (blue), respectively. Note that SMA, Rac1, and Cdc42-over-expressing fibroblasts display an increased number of stress fibers, as compared with vector-alone control cells.

FIG. 8. Cdc42-Overexpression Promotes the Activation of NFkB, Induces Increased Autophagy and Glycolytic Metabolism.

(A) GTPases are strong activators of the transcription factor NFkB. As shown in figure, note that immuno-blot analysis of control or SMA-, Rac1- and cdc42-over-expressing fibroblasts reveals that p-NFkB protein levels are significantly increased, exclusively in Cdc42 fibroblasts, as compared with control fibroblasts.

(B) To validate that Cdc42 induces an autophagic program, cells were subjected to immuno-blot analysis using several autophagy markers. β-actin was used as an equal loading control. Note that Cdc42 increases the expression of the autophagy markers, such as Beclin-1, BNIP3, LAMP-1 and Cathepsin B (37 kDA).

(C) Cdc42 over-expressing fibroblasts display a predominantly glycolytic metabolism, as demonstrated by increased L-Lactate production, under hypoxic conditions. Cells cultured for 48 hours under hypoxic conditions (0.5% O₂) were treated with or without metformin (1 mM), and the results are expressed as ratio between treated versus untreated cells.

(D) The shift towards a predominantly glycolytic metabolism is also demonstrated by decreased mitochondrial activity, as visualized using MitoTracker. Note that Cdc42 significantly decreases mitochondrial activity, as compared with vector alone control or SMA over-expressing fibroblasts. MitoTracker (red); nuclei/DAPI (blue). Original magnification, 60×.

FIG. 9. Cdc42 Over-Expressing Fibroblasts Promote Tumor Growth in Vivo.

We used a xenograft model employing MDA-MB-231 breast cancer cells injected into the flanks of athymic nude mice. MDA-MB-231 breast cancer cells were co-injected with either the empty vector (Lv-105), SMA- or Cdc42-over-expressing fibroblasts.

(A) Comparative trend measurements for tumor growth (days 7-25 post-injection). Tumor volumes were measured with calipers about twice a week and mean tumor volume is plotted versus time, for each experimental group.

(B) Tumor growth. Tumor volumes were also measured at 4 weeks post-injection. Note that fibroblasts over-expressing Cdc42 significantly promote tumor growth, resulting in a L75-fold increase in tumor volume. p=0.01; n=10 tumors per experimental group.

(C) Tumor Angiogenesis. Tumor frozen sections were cut and immuno-stained with anti-CD31 antibodies. Then, vascular density (number of vessels per field) was quantified. The observed 25% increase in tumor angiogenesis in Cdc42 tumors is not sufficient to account for a near two-fold increase in tumor growth. Instead, metabolic reprogramming of the tumor microenvironment, towards L-lactate production, is a more likely mechanism.

FIG. 10. MSF Expression in Cancer-Associated Fibroblasts Drives Tumor Growth.

MSF leads to the up-regulation of SMA and a number of other myo-fibroblast marker proteins, conferring a myo-fibroblast phenotype. The fibroblast activation by MSF is probably induced by TGFβ signaling and the increased expression of the small GTPase Cdc42, driving the activation of NFkB that, in turn, induces autophagy, mitophagy and aerobic glycolysis, thereby promoting tumor growth.

### DEFINITIONS

A pharmaceutical composition typically comprises an active agent and a pharmaceutically acceptable carrier. Pharmaceutically acceptable carriers are well known to those skilled in the art and include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as Ringer's dextrose, those based on Ringer's dextrose, and the like. Fluids used commonly for i.v. administration are found, for example, in Remington: The Science and Practice of Pharmacy, 20.sup.th Ed., p. 808, Lippincott Williams & Wilkins (2000). Preservatives and other additives may also be present, such as, for example, antimicrobials, antioxidants, chelating agents, inert gases, and the like.

As used here, a “nucleic acid” can be DNA, RNA, or a variant thereof. Nucleic acids include, for example, RNAi and antisense molecules.

As used herein, “subject” shall mean any animal, such as a human, non-human primate, mouse, rat, guinea pig or rabbit.

As used herein, “treating” a subject afflicted with a tumor shall mean slowing, stopping or reversing the tumor's progression. In the preferred embodiment, treating a subject afflicted with a tumor means reversing the tumor's progression, ideally to the point of eliminating the tumor itself.

As used herein, “inhibiting the MSF/Cdc42/NFkB cascade” shall mean reducing the activity and/or amount of one or more of MSF, Cdc42 and NFkB.

Therapeutic agents can be administered by any known means including, for example, orally, intravenously, intramuscularly, topically and subcutaneously.

What is claimed is:

1. A method for treating a subject afflicted with a tumor comprising administering to the subject a therapeutically effective amount of an agent that inhibits the MSF/Cdc42/NFkB cascade in the subject's tumor-associated fibroblasts.

2. The method of claim 1, wherein the agent inhibits the MSF/Cdc42/NFkB cascade by inhibiting the activity of one or more of MSF, Cdc42 and NFkB in the subject's tumor-associated fibroblasts.

3. The method of claim 2, wherein the agent inhibits the activity of MSF in the subject's tumor-associated fibroblasts.

4. The method of claim 3, wherein the agent inhibits the activity of MSF in the subject's tumor-associated fibroblasts by interacting with the 10-amino acid residue C-terminal portion of MSF.

5. The method of claim 2, wherein the agent inhibits the activity of Cdc42 in the subject's tumor-associated fibroblasts.

6. The method of claim 2, wherein the agent inhibits the activity of NFkB in the subject's tumor-associated fibroblasts.

7. The method of claim 2, wherein the agent is an antibody, a polypeptide or a small molecule.

8. The method of claim 1, wherein the agent inhibits the MSF/Cdc42/NFkB cascade by inhibiting the expression of one or more of MSF, Cdc42 and NFkB in the subject's tumor-associated fibroblasts.

9. The method of claim 8, wherein the agent inhibits the expression of MSF in the subject's tumor-associated fibroblasts.

10. The method of claim 8, wherein the agent inhibits the expression of Cdc42 in the subject's tumor-associated fibroblasts.

11. The method of claim 8, wherein the agent inhibits the expression of NFkB in the subject's tumor-associated fibroblasts.

12. The method of claim 8, wherein the agent is a nucleic acid.

13. The method of claim 1, wherein the subject is human.

14. The method of claim 1, wherein the tumor is located in one or more of the breast, skin, kidney, lung, pancreas, rectum, colon, prostate, bladder, epithelial tissue and non-epithelial tissue.

15. The method of claim 1, wherein the agent is administered in the form of a pharmaceutical composition.

16. An immortalized cell line that overexpresses MSF.

17. The cell line of claim 16, wherein the cell line is an hTERT-immortalized fibroblast cell line.
